(19) United States
(12) Patent Application Publication
(10) Pub. No.: US 2023/0165749 A1
(43) Pub. Date: Jun. 1, 2023

(54) DEVICES AND METHODS FOR COMFORTING AN INFANT

(71) Applicant: LOMA LINDA UNIVERSITY HEALTH, Loma Linda, CA (US)

(72) Inventors: Erin Hoch, Loma Linda, CA (US); Janel Duggins, Loma Linda, CA (US)

(73) Assignee: LOMA LINDA UNIVERSITY HEALTH, Loma Linda, CA (US)

(21) Appl. No.: 17/997,303

(22) PCT Filed: Apr. 27, 2021
(86) PCT No.: PCT/US21/70462

§ 371 Date: Oct. 27, 2022

Related U.S. Application Data
(60) Provisional application No. 63/015,729, filed on Apr. 27, 2020.

(51) Int. Cl. A61H 23/00; A41B 13/06; A61F 7/03; A61N 5/06; A61H 23/02

(52) U.S. Cl. CPC A61H 23/006; A41B 13/06; A61F 7/03; A61N 5/0625; A61H 23/02; A41B 2400/32; A61N 2005/0659; A61N 2005/0645; A61H 2201/1647; A61H 2205/081; A61H 2205/086; A61H 2201/0107; A61H 2201/0207; A61H 2201/0214; A61H 2201/0278; A61H 2201/0221; A61H 2205/083; A61H 2205/12; A61H 2201/10; A61H 2201/165

(57) ABSTRACT

Described herein are devices and methods used for providing comfort to an infant, and in particular, to devices and methods for introducing a therapy protocol to an infant in, for example, a neonatal intensive care unit.

### CROSS-REFERENCE TO RELATED APPLICATION

This application claims the benefit of and priority to U.S. Provisional Application No. 63/015,729, filed on Apr. 27, 2020, which is incorporated by reference herein in its entirety.

### FIELD OF DISCLOSURE

This disclosure relates generally to a device for providing comfort to an infant, and in particular, to devices and methods for introducing a treatment protocol to an infant in, for example, a neonatal intensive care unit.

### BACKGROUND

Generally, newborn infants or small children may be afflicted with different distressing ailments, such as, for example, colic, acid reflux, drug withdrawal, digestive issues, difficulty eating or sleeping, transitioning from the intrauterine environment, etc. In some cases, the infants may be prone to these conditions for extended periods of time, which may include extended periods of crying. There are limited ways of easing the child's discomfort. Frequently, the child may be hugged or swaddled, along with receiving pats or rubbing to their back sides. However, for infants in, for example, a neonatal intensive care unit, the child may be suffering from a condition that requires comforting for excessive periods of time; however, nurses are often limited in the amount of time they can spend comforting the child.

### SUMMARY

Inventors have recognized the need for devices and methods that can provide comfort to a child in distress as described above. In one aspect, the disclosure is related to a device to provide comfort to an infant. The device includes a body panel sized and shaped to correspond to at least a portion of the infant's torso or midsection, a first arm extending laterally from the body panel, and a second arm extending laterally from the body panel opposite the first arm. The body panel includes a vibration module. At least one of the first arm or the second arm includes a fastening mechanism configured to secure the device about the infant's midsection, where at least one of the first arm or the second arm includes a receptacle or pocket configured to receive a thermal module.

In another aspect, the invention relates to a device to provide comfort to an infant. The device includes a body panel sized and shaped to correspond to at least a portion of the infant's torso or midsection, a first arm extending laterally from the body panel, and a second arm extending laterally from the body panel opposite the first arm. The body panel includes a receptacle or pocket configured to receive at least one of a vibration module or a thermal module, or both. At least one of the first arm or the second arm includes a fastening mechanism configured to secure the device about the infant's midsection. At least one of the first arm or the second arm includes a receptacle or pocket configured to receive at least one of a second vibration module or a second thermal module, or both.

In various embodiments of the foregoing aspects, the vibration module is configured to provide a patting or massaging motion to the infant's bottom. The vibration module can be either permanently or removably attached to the body panel. The vibration module may have a range of operational parameters. In some embodiments, the vibration module is configured to deliver a soft to moderate patting motion to the infant's back or bottom. The patting intensity may range from about 40 to 400 pats per minute, preferably about 150 to 300 pats per minute, and more preferably, about 240 pats per minute. In various embodiments, the patting motion may be delivered to the infant for about 5 to 30 minutes, preferably about 10 to 20 minutes, and more preferably about 15 minutes. In some embodiments, the patting motion may be manually started and stopped or restarted. In some cases, different vibration modules may be provided, each having different operating parameters, so that the device can be customized to a particular application (e.g., infants of different ages/sizes, different conditions to be treated, desired frequency and duration, etc.).

In additional embodiments, the thermal module may be removably or permanently disposed within the pocket and can be either a warming element or a cooling element. In certain embodiments, the thermal module may be a disposable, chemical activated warming element, an infrared warming element, or similar. The pocket(s) may be located on the first arm, the second arm, or both to suit a particular application. Typically, the pocket(s) will be positioned to correspond to the infant's abdominal area when the arms are in the secured configuration. This allows for a thermal (and/or vibration) module to provide a therapeutic action to the child's digestive system. The various pockets disclosed herein can include one or more fastening mechanisms to secure the module therein. The fastening mechanisms can include hook and loop fasteners, buttons, snaps, buckles, zippers, etc. as would be known to those in the apparel and/or medical clothing industry.

In additional embodiments, the device includes a limb portion that extends longitudinally from a lower portion of the body panel. Typically, the limb portion is sized and shaped to at least partially envelop the infant's feet. Alternatively or additionally, a limb portion could be provided that extends longitudinally from an upper portion of the body panel. In this case, the upper limb portion may be sized and shaped to partially envelop the child's head to, for example, provide warmth or facilitate the delivery of phototherapy (e.g., a special light source within a hood). The limb portion(s) may also include one or more pockets configured to receive at least one of a thermal module or a vibration module to, for example, heat the infant's heels prior to a needle stick. In some embodiments, the lower limb portion can be manufactured in layers, such that a receptacle is formed into which the child's feet and legs can be inserted.

In further embodiments of the device, the body panel, the first and second arms, and the limb portions can be made of, for example, a soft fabric (natural or synthetic fibers) that may be disposable or washable and/or a polymeric material. In some embodiments, the body panel may be made of a soft, absorbent fabric (either disposable or washable), while the arms may be made of a transparent or translucent polymeric material that makes it easier for the infant's abdominal area to be monitored. For example, the arms may be made of a soft vinyl or silicone and/or include a gel material (or a gel infused fabric). In certain embodiments, the device materials are configured to allow light from a phototherapy device to pass therethrough. In additional embodiments, the device may include a controller electrically connected to the device (e.g., the vibration module and/or the thermal module) so as to provide power and exchange one or more signals with the device. The device may also include a power source, such as a battery or electrical cord. In some embodiments, the controller communicates wirelessly with the device, while in others it may be disposed within the device, for example, in the body panel.

The device may also include one or more sensors (e.g., a pulse oximeter, thermal sensor, pressure sensor, position/proximity sensor, motion sensor, electrocardiogram sensor, heart rate sensor, electroencephalogram sensor, electromyogram sensor, respiration rate sensor, visual/imaging sensor, etc.) for monitoring and/or determining a condition of the infant (heart rate, blood pressure, temperature, movement, etc.) and activating a treatment protocol (e.g., vibration or heating or both), as needed, or in some cases activating an alarm. In some embodiments, the one or more sensors is disposed on the body panel; however, sensors can also be located on the arms or limb portions to suit a particular application. The device may also include a light source configured to deliver phototherapy to the infant. In some embodiments, the body panel may include additional pocket(s) for introducing a thermal module thereto (e.g., in addition to or instead of the vibration module).

In yet another aspect, the invention relates to a method of providing comfort to an infant by utilizing any of the devices described herein. The method may include the steps of positioning the infant relative to the body panel of the device, securing the device about a portion of the infant with the arms of the device, and initiating a treatment protocol to comfort the infant. The treatment protocol may include exposing the infant to at least one of vibrational, thermal or photo therapy, where the therapy may be applied to, for example, at least one of the infant's back side, abdominal area, or feet. For example, the device can be used to apply vibrations to the infant's back side, while applying thermal therapy to the infant's abdominal area. The treatments can be automatically or manually initiated and can run indefinitely of for preset time periods.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Accordingly, these and other objects, along with advantages and features of the present disclosure herein disclosed, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the disclosure and are not intended as a definition of the limits of the disclosure. For purposes of clarity, not every component may be labeled in every drawing. In the following description, various embodiments of the present disclosure are described with reference to the following drawings, in which:

FIG. 1 is a schematic plan view of a device for comforting an infant in accordance with one or more embodiments of the disclosure;

FIG. 2 is a schematic plan view of the device of FIG. 1 in a secured configuration in accordance with one or more embodiments of the disclosure;

FIG. 3 is a schematic plan view of an alternative device for comforting an infant in accordance with one or more embodiments of the disclosure;

FIG. 4 is a schematic plan view of the device of FIG. 3 in a secured configuration in accordance with one or more embodiments of the disclosure; and

FIG. 5 is a schematic plan view of a device in use with an infant in a secured configuration in accordance with one or more embodiments of the disclosure.

### DETAILED DESCRIPTION

Reference will now be made to the exemplary embodiments illustrated in the drawings, and specific language will be used here to describe the same. It will nevertheless be understood that no limitation of the scope of the disclosure is thereby intended. Alterations and further modifications of the inventive features illustrated herein, and additional applications of the principles of the disclosure as illustrated herein, which would occur to one skilled in the relevant art and having possession of this disclosure, are to be considered within the scope of the disclosure.

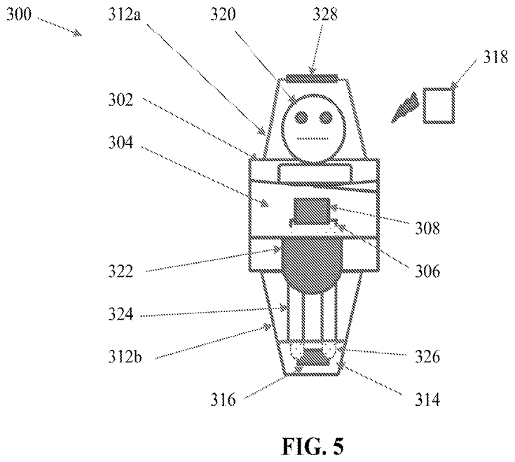

FIGS. 1 and 2 depict one embodiment of a device 100 for comforting an infant. As shown, the device 100 includes a body panel 102 that is sized and shaped so as to generally correspond to the infant's midsection or torso (e.g., 322 in FIG. 5). Generally, the device can be sized for a micro-preemie, a preemie, a term baby, or a neonatal/infant. In some embodiments, the body panel 102 has a substantially rectangular and planar shape; however, the panel 102 can be contoured for a particular application (e.g., to accommodate infants of varying size, the need to interface with or accommodate various body parts, etc.). The body panel 102 includes a vibration module 106 disposed therein to provide a patting or massaging motion to the infant's back side. In some embodiments, the vibration module is permanently attached to the body panel 102, while in others it is removable, for example, via a pocket on the panel 102 that allows for interchanging the vibration module 106 with another vibration module and/or a thermal module.

The device 100 includes two arms 104a, 104b (collectively 104) that extend outwardly from the sides of the body panel 102. The arms 104 can be formed as part of the body panel 102 or attached there to by, for example, a hook and loop fastener (Velcro®), snaps, or stitching to suit a particular application (e.g., permanent vs. removably attached, adjustability, etc.). Typically, the arms 104 are planar and relatively soft panels that can be wrapped about the infant's midsection to secure the device 100 thereto, as shown in FIG. 2. The arms 104 are roughly rectangular in shape, but can be any size and shape as needed to accommodate the infant's anatomy. As shown in FIG. 2, the two arms 104 overlap and can be secured to one another via an attachment mechanism 110, such as a hook and loop fastener (Velcro®), buttons, snaps, buckles, draw strings, zippers, or other mechanisms as would be known to those in the apparel and/or medical clothing industry. In one embodiment, the fastening mechanism includes mating hook and loop fasteners 110a, 110b that cover a significant portion (e.g., about 50% or more of the length of the arms) such that the fit of the arms in the secured configuration can be adjusted to accommodate infants of different sizes. In certain embodiments, only a single arm 104 is required, and the arm is configured to be secured around the infant and to the opposite side of the body panel 102.

The device 100 may also include one or more thermal modules 108. These modules 108 can be permanently or removably attached to the device 100 depending on the application. As shown in FIG. 1, the thermal module 108 is disposed on one of the arms 104a, such that when the infant is secured within the device, the thermal module 108 is oriented proximate the infant's abdominal area. In some embodiments, the thermal module 108 may be disposed on the other arm 104b or both arms 104 to suit a particular application. In various embodiments, the thermal module may be a warming element or a cooling element, either a reusable or a disposable type. In certain embodiments the thermal module 108 is a disposable chemical activated warming element, such as an air-activated heat pack as available from HotHands® that can be easily replaced when it is no longer effective. In certain embodiments, the thermal module 108 can include an infrared heater powered through the device 100 or directly via another power source.

Also shown in FIGS. 1 and 2 is a controller 118 that may be wired or wirelessly connected to the device 100 to send and receive signals representative of, for example, sensing the infant's movement and initiating the vibration module in response thereto. In particular, the controller 118 may be configured to receive data from one or more sensors 130 built into the device 100 and be programmed to initiate a treatment protocol, such as vibrate for 10 minutes, off for 2 minutes, on for 10 minutes, etc., and/or energize a thermal module (e.g., an infrared heater), as would be understood by those skilled in the art. In some embodiments, the device may be manually started either directly (e.g., by actuating a switch on the device) or through the controller 118 (e.g., a nurse could turn the device 100 on for an indefinite period or a preset time period. The controller 118 may include a power source, such as a battery or electrical cord that can supply power to the device 100.

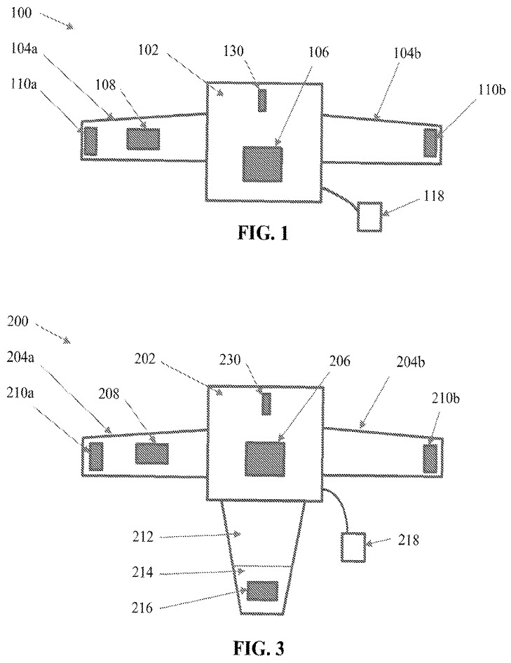

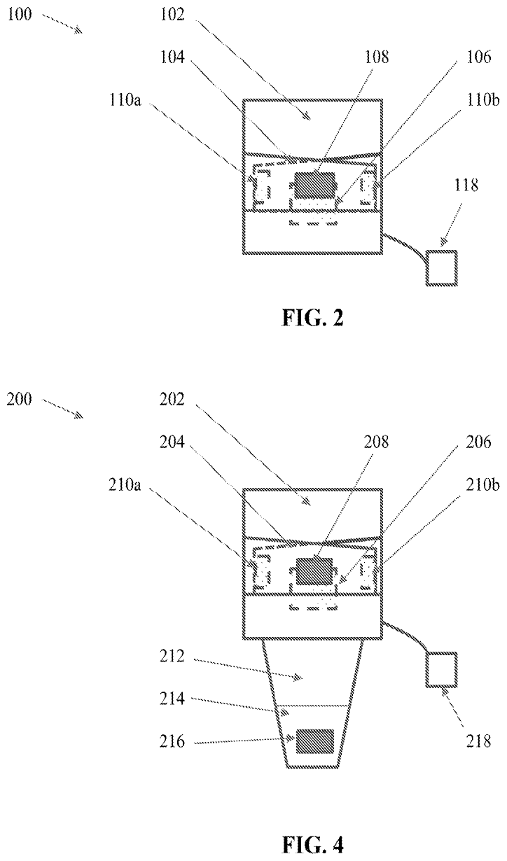

FIGS. 3 and 4 depict an alternative device 200 that is substantially similar to device 100 insofar as it includes a body panel 202, arms, 204a, 204b, a vibration module 206, thermal modules 208, sensors 230, and a controller 218 as described above. The device 200 of FIGS. 3 and 4 differs from device 100 at least because of the addition of a limb portion 212 that extends longitudinally from a bottom edge of the body panel 202. Alternatively or additionally a limb portion may extend longitudinally from a top edge of the body panel (see FIG. 5). The limb portion 212 has a generally elongate shape that corresponds to the infant's lower limbs and may at least partially envelop the infant's feet (see FIG. 5). In some embodiments, the limb portion 212 includes a receptacle 214 for receiving the infant's feet. For example, two layers of material may be stitched along the perimeter thereof, leaving one edge open to form the receptacle 214. The limb portion 212 may also include a thermal module 216, either permanently or removably attached to the limb portion 212. In some cases, the thermal module 216 can be a warming element as described herein for warming the infant's heels to, for example, improve blood flow. Similar to FIGS. 1 and 2, FIGS. 3 and 4 depict the device 200 in an open configuration (FIG. 3) and a secured configuration (FIG. 4).

FIG. 5 depicts yet another alternative embodiment of the device 300 similar to those described above, including a body panel 302, arms 304, vibration module 306, thermal modules 308, 316, limb portions 312a, 312b, and a controller 318 wireless connected to the device 300. FIG. 5 also depicts a representative infant 320 secured within the device 300. As can be seen, the arms 304 secure the infant 320 about its midsection 322 and the infant's legs 324 extend along the lower limb portion 312b with the feet 326 disposed within the receptacle 314. The upper limb portion 312a generally corresponds to the infant's head and be configured as a hood to keep the infant 320 warm and/or may incorporate a specialized light source 328 for delivering phototherapy to the infant 320 (e.g., for the treatment of jaundice).

Having now described some illustrative embodiments of the disclosure, it should be apparent to those skilled in the art that the foregoing is merely illustrative and not limiting, having been presented by way of example only. Numerous modifications and other embodiments are within the scope of one of ordinary skill in the art and are contemplated as falling within the scope of the disclosure. In particular, although many of the examples presented herein involve specific combinations of method acts or system elements, it should be understood that those acts and those elements may be combined in other ways to accomplish the same objectives.

Furthermore, those skilled in the art should appreciate that the parameters and configurations described herein are exemplary and that actual parameters and/or configurations will depend on the specific application in which the systems and techniques of the disclosure are used. Those skilled in the art should also recognize or be able to ascertain, using no more than routine experimentation, equivalents to the specific embodiments of the disclosure. It is, therefore, to be understood that the embodiments described herein are presented by way of example only and that, within the scope of any appended claims and equivalents thereto; the disclosure may be practiced other than as specifically described.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to any claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish claim elements.

What is claimed is:

1. A device to provide comfort to an infant, the device comprising:
a body panel sized and shaped to correspond to at least a portion of the infant's torso, wherein the body panel comprises a vibration module;
a first arm extending laterally from the body panel; and
a second arm extending laterally from the body panel opposite the first arm, wherein:
at least one of the first arm or the second arm comprises a fastening mechanism configured to secure the device about a midsection of the infant; and
at least one of the first arm or the second arm comprises a pocket configured to receive a thermal module.

2. A device to provide comfort to an infant, the device comprising:
a body panel sized and shaped to correspond to at least a portion of the infant's torso, wherein the body panel comprises a receptacle configured to receive at least one of a vibration module and a thermal module;
a first arm extending laterally from the body panel; and
a second arm extending laterally from the body panel opposite the first arm, wherein:
at least one of the first arm or the second arm comprises a fastening mechanism configured to secure the device about a midsection of the infant; and
at least one of the first arm or the second arm comprises a pocket configured to receive at least one of a second vibration module and a second thermal module.

3. The device of claim 1 or 2, wherein the vibration module is configured to provide a patting or massaging motion to the infant's back or bottom.

4. The device of claim 1 or 2, wherein the vibration module is removably attached to the body panel.

5. The device of claim 1 or 2, wherein the vibration module operates at about 240 pats per minute for up to 15 minutes.

6. The device of claim 1 or 2, further comprising a thermal module disposed within the pocket, wherein the thermal module comprises at least one of a warming element or a cooling element.

7. The device of claim 6, wherein the thermal module comprises a disposable, chemical activated warming element.

8. The device of claim 6, wherein the thermal module comprises an infrared warming element.

9. The device of claim 6, wherein the pocket comprises a fastening mechanism configured to secure the thermal module therein.

10. The device of claim 6, wherein the pocket is disposed on the at least one of the first arm or the second arm so as to position the thermal module proximate the infant's abdominal area when the arms are in a secured configuration.

11. The device of claim 1 or 2, further comprising a limb portion extending longitudinally from a lower portion of the body panel, wherein the limb portion is sized and shaped to at least partially envelop the infant's feet.

12. The device of claim 11, wherein the limb portion comprises a pocket configured to receive at least one of a thermal module or a vibration module.

13. The device of claim 11, wherein the body panel, the first and second arms, and the limb portion comprise at least one of a fabric configured to a phototherapy treatment to pass therethrough, a soft, clear plastic, or a gel material.

14. The device of claim 1 or 2, further comprising a controller electrically connected to the device so as to provide power thereto and exchange one or more signals with the device.

15. The device of claim 14, further comprising one or more sensors for determining a condition of the infant, wherein the sensors generate a signal to the controller and the controller activates a treatment protocol in response thereto.

16. The device of claim 15, wherein the one or more sensors is disposed on the body panel.

17. The device of claim 1 or 2, further comprising a light source configured to deliver phototherapy to the infant.

18. The device of claim 1 or 2, further comprising a power source.

19. The device of claim 1 or 2, wherein the body panel further comprises a pocket for introducing a thermal module in addition to or instead of the vibration module.

20. A method of providing comfort to an infant by utilizing the device of claim 1 or 2, wherein the method comprises the steps of:
positioning the infant relative to the body panel of the device;
securing the device about a portion of the infant with the arms of the device; and
initiating a treatment protocol to comfort the infant.

21. The method of claim 20, wherein the treatment protocol comprises exposing the infant to at least one of vibrational, thermal or photo therapy.

22. The method of claim 21, wherein the therapy is applied to at least one of the infant's back side, abdominal area, or feet.
